(19) United States
(12) Patent Application Publication
(10) Pub. No.: US 2024/0115286 A1
(43) Pub. Date: Apr. 11, 2024

(54) RF SHAVER AUXILIARY SUCTION

(71) Applicant: GYRUS MEDICAL LIMITED, Cardiff (GB)

(72) Inventor: James Alan DICKSON, Cardiff (GB)

(73) Assignee: GYRUS MEDICAL LIMITED, Cardiff (GB)

(21) Appl. No.: 18/376,950

(22) Filed: Oct. 5, 2023

Related U.S. Application Data
(60) Provisional application No. 63/413,852, filed on Oct. 6, 2022.

(51) Int. Cl. A61B 17/32; A61B 18/12; A61B 18/14

(52) U.S. Cl. CPC A61B 17/32002; A61B 18/1206; A61B 18/148; A61B 2218/007

(57) ABSTRACT

The present disclosure relates to a rotary shaver arrangement for a surgical instrument, the rotary shaver arrangement comprising an outer tubular member; and an inner tubular member rotatably mounted in a central passageway of the outer tubular member, the inner tubular member providing a central suction lumen. Wherein the inner tubular member further comprises one or more longitudinal grooves that run from the distal end to the proximal end of the rotary shaver arrangement to create a suction lumen passageway between the inner tubular member and the outer tubular member. This rotary shaver arrangement optimises the balance between shaver performance and RF thermal performance, by providing a defined minimum gap between the inner and outer tubular members in the form of longitudinal grooves, to provide an additional pathway for saline used to cool the RF suction lumen.

### TECHNICAL FIELD

The present disclosure generally relates to surgical devices, and in particular to a rotary shaver arrangement for a surgical instrument, an end effector for an electrosurgical instrument, an electrosurgical instrument, and an electrosurgical system.

Specifically, the present disclosure relates to suction flow pathways in electrosurgical instruments. More specifically, the present invention relates to additional suction flow pathways located in end effectors of electrosurgical instruments.

### BACKGROUND AND RELATED ART

Surgical instruments, including radio frequency (RF) electrosurgical instruments, have become widely used in surgical procedures where access to the surgical site is restricted to a narrow passage, for example, in minimally invasive “keyhole” surgeries. Electrosurgical instruments provide advantages over traditional surgical instruments in that they can be used for coagulation and tissue sealing purposes. Surgical apparatus used to shave, cut, resect, abrade and/or remove tissue, bone and/or other bodily materials are known.

In some electrosurgical instruments, e.g., shaver instruments, the instrument can include a mechanical cutting surface, such as a rotating blade disposed on an elongated inner tubular member that is rotated within an elongated outer tubular member having a cutting window. The inner and outer tubular members together form a surgical cutting instrument or unit. In this application, the inner and outer tubular members are also referred to as inner and outer blades. In general, the elongated outer tubular member includes a distal end defining an opening or cutting window disposed at a side of the distal end of the outer tubular member. The cutting window of the outer tubular member exposes the cutting surface of the inner tubular member (located at a side of the distal end of the inner tubular member) to tissue, bone and/or any other bodily materials to be removed. A powered handpiece is used to rotate the inner tubular member with respect to the outer tubular member while an outer tubular member hub (connected to the proximal end of the outer tubular member) is fixed to the handpiece and an inner tubular member hub (connected to the proximal end of the inner tubular member) is loosely held in place by the powered handpiece.

In some instruments the inner tubular member is hollow and has a cutting window on a side surface of its distal end such that tissue, bone, etc. will be cut or shaved as the cutting window of the inner tubular member aligns with and then becomes misaligned with the cutting window of the outer tubular member as the inner tubular member is rotated within the outer tubular member. In this regard, it can be said that the cutting device removes small pieces of the bone, tissue, etc. as the inner tubular member is rotated within the outer tubular member.

In some instruments a vacuum is applied through the inner tubular member such that the bodily material that is to be cut, shaved, etc. is drawn into the windows of the inner and outer tubular members when those windows become aligned, thereby facilitating the cutting, shaving, etc. of the tissue, which then travels through the inner tubular member due to the suction. It is also known to supply an irrigation fluid, which can include a liquid, to the surgical site via a passage provided between the inner and outer tubular members.

Some instruments, e.g., wet-field RF hand instruments for arthroscopy, use a saline suction pathway at the distal tip during either ablating or coagulating tissue. In general terms, suction provides the following benefits:
(i) Cools the RF tip by drawing colder saline over the hot RF tip during use;
(ii) Helps to remove ablated tissue debris from surgical site;
(iii) Removes bubbles to improve joint visibility; and
(iv) Has a positive effect on the formation of plasma at the tip.

Many times during surgery, the surgeon wishes to apply RF energy to either coagulate bleeding vessels, or ablate tissue in the surgical site without performing cutting with a shaver instrument. This usually is done by withdrawing the shaver instrument and inserting a dedicated RF ablation/coagulation/suction device (for example, a RF wand which is a tube to which suction is applied). However, exchanging the surgical tool for the dedicated RF wand is time-consuming. Furthermore, insertion and removal of instruments into the patient can cause trauma and irritation to the tissue of the patient, and thus it is desirable to minimize the number of times that surgical instruments need to be withdrawn and inserted/reinserted into the patient.

An RF shaver has both shaving and RF energy capabilities. In RF shaver electrosurgical instruments, the shaver side (the side on which the tissue, bone, etc. will be mechanically cut or shaved as the cutting window of the inner tubular member aligns with and then becomes misaligned with the cutting window of the outer tubular member as the inner tubular member is rotated within the outer tubular member) is opposite an RF side which has an electrode assembly with an active electrode for tissue treatment. The RF side is typically provided with a suction aperture.

An RF Shaver with opposite sided functionality (RF on one side, shaver on the other) must balance the RF functionality with the shaver functionality at the distal tip of the device.

In this configuration, hot saline is created at the active tip and pulled via a negative pressure source into the distal tip assembly. In this assembly, the inner blade is usually closed during RF use.

The hot saline then travels down a suction lumen. Due to the conflicting requirements between shaver performance and RF thermal performance, an optimum shaft configuration is difficult to achieve. In brief, in an ideal shaver-only configuration (ie without an RF element) the inner shaft suction lumen is as large as possible in order to deal with tissue fragments created by the shearing action in the substantially larger blade cutting window. The inner bore of the inner blade therefore becomes the smallest constriction in the suction system, and can therefore be prone to blockage.

Conversely, the ideal RF-only (ie without a mechanical shaver) suction lumen has to carry away ablated tissue fragments, but these fragments are typically much smaller because the hole in the RF tip precludes large chunks of tissue from getting into the suction lumen. Therefore, the active tip or ceramic insulator are the smallest constrictions in the suction system when RF is used with the blade window closed, and the suction lumen in the shaft only has to be marginally larger to carry away tissue debris. The advantage of this smaller suction lumen in an RF device is that it allows space in the overall device construction to create an air gap between the inner suction pathway, which contains heated saline and the outer shaft which could be in direct patient contact and needs to be cooler.

As detailed above, a conflict therefore arises when trying to create a ‘best-of-both’ RF shaver, which needs a large suction lumen to maintain shaver performance, and also an air gap for thermal insulation during RF ablation use. An obvious solution is to simply increase the outer shaft outside diameter, but this has disadvantages for the user such as poor tissue access and reduced visibility. The patient also suffers through larger incisions and cannula required.

Therefore, one problem to be solved is that in a dual-sided RF shaver, there exists a hot saline pathway which could be poorly insulated from the outer shaft by a small air gap due to the access requirements of such a device and limits on the outside diameter thereof. A configuration is therefore required which maintains high shaver performance levels while increasing the cooling capabilities of the device, in order to reduce the temperature of the suction lumen and inner tubular member or blade.

### SUMMARY OF THE INVENTION

Embodiments of the present invention provide a RF shaver arrangement comprising both mechanical shaving and RF energy capabilities with opposite side functionality i.e. RF on one side and shaver on the other. Specifically, the present disclosure relates to optimising the balance between maintaining high shaver performance levels and also maximising thermal insulation of the device during RF ablation use as the device could be in direct contact with the patient. The present disclosure achieves this by providing a defined minimum gap in the form of longitudinal grooves in the RF shaver arrangement which carry cool saline between the inner and outer shafts during RF use. The longitudinal grooves are formed within the inner blade and/or the stator of the RF shaver. The longitudinal grooves provide a passageway to improve/facilitate the improved cooling of the RF suction lumen on the RF device whilst maintaining high performance of the shaver as the overall large inner bore and tight clearances required for effective shaving are unaffected.

The present disclosure incorporates the above design characteristics to create an additional cooling pathway between the inner and outer shafts to create a cooling jacket of saline on the outside of the hot inner shaft during RF ablation/coagulation.

The present disclosure has the advantages of increasing the effectiveness of the cooling of the outer shaft, due to the presence of the additional cooling pathway, to aid in the cooling of both the inner and outer shafts which get hot during RF ablation/coagulation. This ensures that any hot shaft surface is less likely to contact the patient and cause discomfort or injury. The solution balances rotary shave performance with the need to cool the RF suction lumen on the surgical instrument. The rotary shave performance is not affected as the diameters of the inner and outer shafts which are required for effective rotary shave performance are unchanged. Therefore, a further advantage is that rotary shave performance is not affected and there is a balance between rotary shave performance and the need to provide cooling suction to the RF elements of the surgical device.

This is in comparison to a legacy RF rotary shaver arrangement where a nominal clearance between the inner and outer shafts or blades generally always exists through the chosen nominal design and tolerances. For every inner blade to fit every outer blade in a defined tolerance interval, the practical gap on legacy RF shaver arrangements will vary between zero and around 50 microns radially. Therefore, legacy RF shaver arrangements have no design intent to create a saline gap or suction lumen in this area, but they create one anyway through the necessity of needing a gap to allow the inner to rotate while shaving tissue. An easy way to increase the gap for an RF shaver, and give better thermal RF performance would be to increase this nominal shaver clearance gap to allow more saline between the inner and outer blades during RF. This solution of simply modifying the inner diameter and outer diameter of the inner blade and stator would lead to very poor cutting performance, as the blade edges would be too far apart to create the shearing action to cut tissue.

In view of the above, from a first aspect the present disclosure provides a rotary shaver surgical instrument, comprising: an outer tubular member having an outer cutting window formed therein; an inner tubular member having an inner cutting window formed therein, the inner tubular member being rotatably arranged within the outer tubular member; at least one of the outer tubular member or the inner tubular member having one or more grooves formed in a surface thereof running from a distal end of the surgical instrument where the inner and outer cutting windows are formed to a proximal end whereby to form in use at least one suction lumen passageway between the inner tubular member and the outer tubular member.

In this respect, the suction lumen thus formed by the one or more grooves provides an additional and deliberate suction path from the proximal end to the distal end of the instrument, which is in addition to any intrinsic suction path that might intrinsically be formed due to any spacing between the inner and outer tubular members that arises dues to nominal design and manufacturing tolerances of the instrument.

Further features and advantages of the present disclosure will be apparent from the appended claims.

### BRIEF DESCRIPTION OF THE DRAWINGS

Examples of the present disclosure are now described with reference to the accompanying drawings, wherein like reference numerals refer to like parts, in which:

FIG. 1 shows a schematic diagram of an electrosurgical system including an electrosurgical instrument;

FIG. 2 shows a perspective view of a dual-sided RF shaver device with longitudinal grooves within the inner blade/shaft.

FIG. 3 shows an inner blade geometry arrangement for multi-purpose blades, e.g. RF shavers;

FIG. 4 shows a partial cross-sectional view of the same dual-sided RF shaver device as FIG. 2;

FIG. 5 shows a side partial cross-sectional view of the same dual-sided RF shaver device as in FIG. 4 and FIG. 2.

FIG. 6 shows a perspective view of a dual-sided RF shaver device with longitudinal grooves within the inner stator illustrating embodiments of the present disclosure;

FIG. 7 shows a view of a dual-sided RF shaver as in FIG. 6, with the inner blade/shaft removed.

FIG. 8 shows a partial cross-sectional view of the same dual-sided RF shaver device as FIG. 6;

FIG. 9 shows a side partial cross-sectional view of the same dual-sided RF shaver device as in FIG. 8 and FIG. 6;

FIG. 10 is a flowchart indicating a reprocessing method for the rotary shaver arrangement.

### DETAILED DESCRIPTION OF THE PREFERRED EMBODIMENTS

The present disclosure relates to an RF shaver comprising both shaving and RF energy capabilities with opposite side functionality i.e. RF on one side and shaver on the other. Hot saline is created at the active tip during RF use and pulled via a negative pressure source into the distal tip assembly. In this assembly, the inner blade is closed during RF use. Specifically, the present disclosure relates to optimising the balance between maintaining high shaver performance levels and also maximising thermal insulation of the device during RF ablation use as the device could be in direct contact with the patient. The present disclosure achieves this by providing a defined minimum gap in the form of longitudinal grooves in the RF shaver arrangement which carry cool saline between the inner and outer shafts during RF use. The longitudinal grooves are formed in the outer edge of the inner blade and/or the inner edge of the stator of the RF shaver. The longitudinal grooves provide a passageway to improve/facilitate the improved cooling of the RF suction lumen on the RF device whilst maintaining high performance of the shaver as the overall large inner bore and tight clearances required for effective shaving are unaffected.

Embodiments of the present disclosure provide a RF rotary shaver arrangement for a surgical instrument, the RF rotary shaver arrangement comprising longitudinal grooves that are long enough to reach from the cool saline in the joint to the area between the outer and inner shafts, where the clearance between the inner and outer shafts becomes large enough to provide a suction lumen all the way to the hub area. The presence of the grooves creates an additional cooling pathway between the inner and outer shafts to create a cooling jacket of saline on the outside of the hot inner shaft during RF ablation/coagulation. The additional cooling pathway ensures that the heat in the inner shaft (which gets hot during RF ablation/coagulation) does not reach the outside surface of the outer shaft. This ensures that no hot shaft outer surface is able to contact the patient and cause discomfort or injury. The solution balances shave performance with the need to cool the RF suction lumen on the surgical instrument. The shave performance is not affected as the diameters of the inner and outer shafts which are required for effective shave performance are unchanged. This is in comparison to a legacy RF rotary shaver arrangement where a nominal clearance between the inner and outer shafts or blades generally always exists through the chosen nominal design and tolerances. For every inner blade to fit every outer blade in a defined tolerance interval, the practical gap on legacy shaver arrangements (including known RF shavers) will vary between zero and around 50 microns radially. Therefore, legacy RF shaver arrangements have no design intent to create a saline gap or suction lumen in this area, but they create one anyway through the necessity of needing a gap to allow the inner to rotate while shaving tissue. An easy way to increase the gap for an RF shaver, and give better thermal RF performance would be to increase this nominal shaver clearance gap to allow more saline between the inner and outer blades during RF. This solution of simply modifying the inner diameter and outer diameter of the inner blade and stator would lead to very poor cutting performance, as the blade edges would be too far apart to create the shearing action to cut tissue.

Further details of an embodiment of the present disclosure will now be described with respect to the figures.

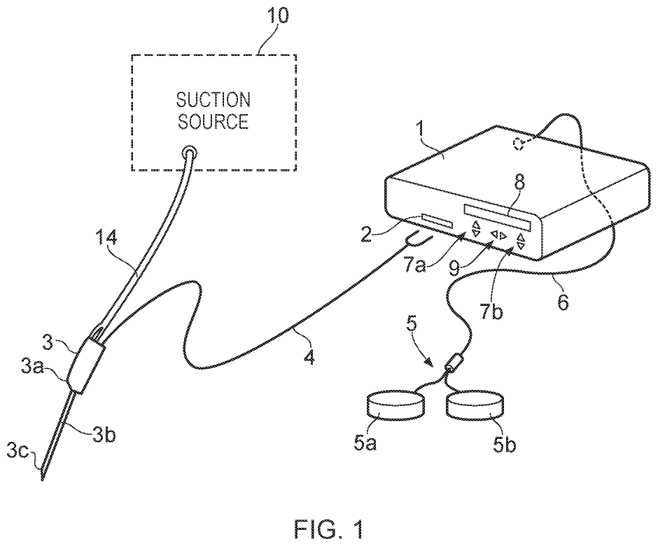

Referring to the drawings, FIG. 1 shows an electrosurgical system including an electrosurgical generator 1 having an output socket 2 providing an RF output, via a connection cord 4, for an electrosurgical instrument 3. The instrument 3 has a suction tube 14 which is connected to suction pump 10. Activation of the generator 1 may be performed from the instrument 3 via a hand-switch (not shown) on the instrument 3, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

The instrument 3 includes a proximal handle portion 3a, a shaft 3b extending in a distal direction away from the proximal handle portion, and a distal end effector assembly 3c at the distal end of the shaft 3b. A power connection cord 4 connects the instrument to the RF generator 1. The instrument may further be provided with activation buttons (not shown), to allow the surgeon operator to activate either the mechanical cutting function of the end effector, or the electrosurgical functions of the end effector, which typically comprise coagulation or ablation.

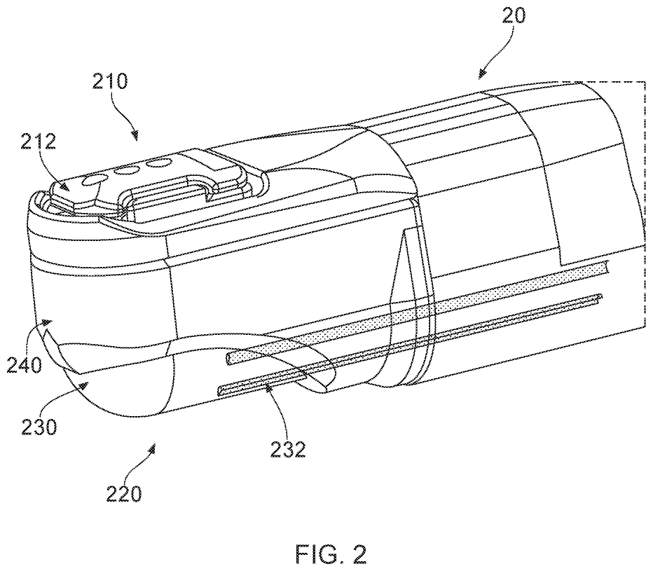

The instrument 3 may be an RF shaver instrument. An example of an RF shaver instrument is shown in FIG. 2. Combining a shaver device with a RF wand is not straightforward. In prior art arthroscopic shavers, the cutting blade geometry and interface between outer blade and inner blade can be optimised for one function only—mechanical engagement and shaving of tissue. Similarly, prior art RF electrodes can optimise their suction pathways to prevent any leakage along the suction lumen and direct all available flow towards the tissue contacting active electrode area. With a dual-sided RF shaver combination device, the suction characteristics must be optimised for both modes. While shaving, the shaver window aperture and relative shearing geometry between the inner and outer blades must allow for effective tissue engagement and suction without allowing too much suction flow or pressure to be lost through the RF suction hole on the opposite side to the shave aperture. For this reason, the RF suction hole is kept as small as viable for RF use. When using the RF ablation mode, the inner blade is parked in a static ‘closed’ position (i.e., there is no overlap between the cutting windows) and all available suction flow should be directed through the RF suction aperture. The optimisation of each of these flow paths ensures that collateral tissue is not dragged into the non-active face of the device. In short, collateral tissue is not dragged towards the shaver aperture while using RF, and visa-versa. This reduces the risk of collateral tissue damage overall.

FIG. 2 shows a dual-sided RF shaver combination device 20. The device has a RF side 210 and a shaver side 220. The RF side 210 comprises an active electrode area 212. In an RF shaver configuration, there are two possible suction flow paths. One through the cutting window aperture on the shaver side 220, and one through an aperture on the RF side 210. It is preferable to minimise the flow through the shave window aperture while using the RF side and minimise flow through the RF side aperture while using the shave function. This ensures that the maximum available saline suction pressure and flow rate is directed through the working suction aperture (whether that is the RF side aperture or the shave window aperture). The shaver suction window can preferably be closed during RF activation, via keeping the inner blade stationary with the teeth overlapping to close the window (i.e., such that there is no overlap between the cutting windows). For optimal suction to be directed through the RF side aperture, the shaver suction window must be closed. For the shaver suction window to be closed, the cutting windows of the inner 230 and outer tubular members 240 need to be rotationally/angularly positioned such that there is no overlap between the two cutting windows.

In this application, as shown in FIG. 2, the inner tubular member 230 (also known as the inner shaft or inner blade) comprises a series of longitudinal grooves 232 that are dug from the inner tubular member 230 to create additional saline cooling pathways between the inner and outer tubular members.

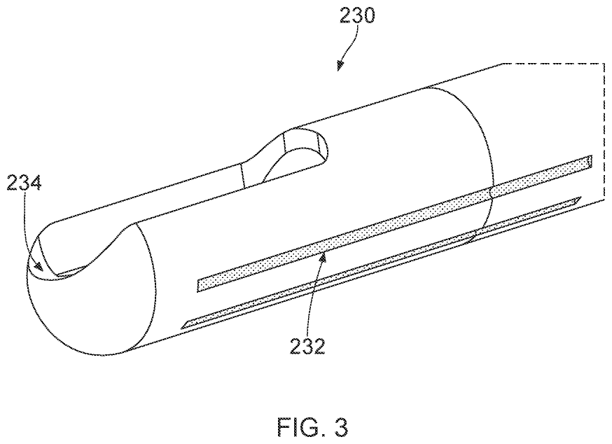

FIG. 3 shows a more detailed view of the geometry of the inner tubular member or inner blade 230. Inner tubular member 230 has a square cut section or notch-shaped region 234 at its distal end which creates an opening. The opening at the distal end creates the cutting window of the inner tubular member. The edges of the cut-out of the inner tubular member form a cutting surface which, when the inner tubular member 230 rotates within the outer tubular member 240, cause tissue, bone, etc. to be cut or shaved as the cutting window of the inner tubular member/blade 230 aligns with and then becomes misaligned with the cutting window of the outer tubular member 240 as the inner tubular member is rotated within the outer tubular member. The cutting surface comprises at least one sharpened edge to form a cutting blade. The cutting blade may be serrated such that it comprises teeth.

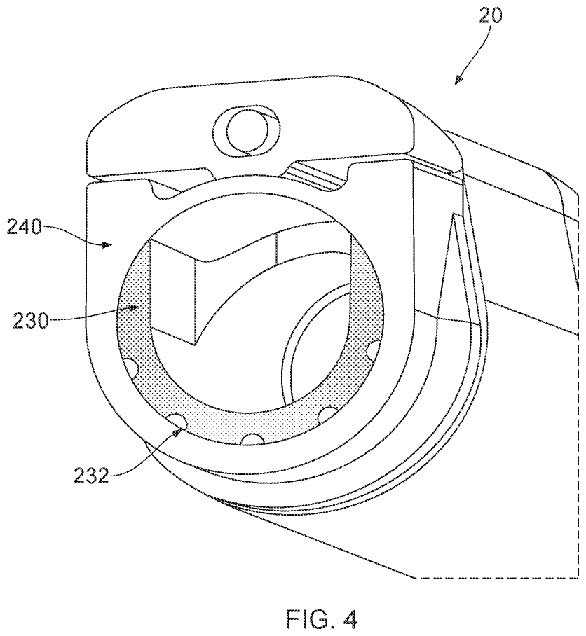

FIG. 4 shows the same dual-sided RF shaver combination device 20 as FIG. 2, but the device is shown in a partial cross-sectional arrangement to better show the longitudinal grooves 232 that are present on the inner tubular member 230. FIG. 4 shows the longitudinal grooves 232 as being dug into the outer surface of the inner tubular member or inner blade 230. Therefore, it can be seen that the longitudinal grooves 232 create passageways between the inner tubular member 230 and the outer tubular member 240, in which cool saline can pass to cool the inner tubular member 230.

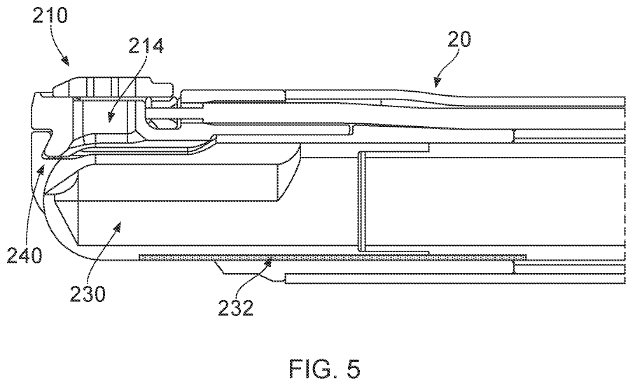

FIG. 5 shows the same dual-sided RF shaver combination device as in FIGS. 2 and 4, but the device is shown in a partial cross-sectional arrangement from the side. It can be seen that the RF side 210 comprises a RF suction pathway 214.

FIGS. 2-5 show an arrangement of the RF shaver arrangement according to a first embodiment. The key design feature of this embodiment is that the inner tubular member or inner blade 230 comprises the longitudinal grooves 232 that create a cool saline passageway in order to cool the inner tubular member 230, that can reach high temperatures as a result of RF ablation/coagulation. In FIGS. 2-5 five longitudinal grooves 232 were depicted for example purposes, it is feasible that there could be more or less longitudinal grooves 232. An advantage of this embodiment is that the longitudinal grooves 232 are positioned on the outer edge of the inner tubular member 230 so various manufacturing options are available such as traditional machining or EDM.

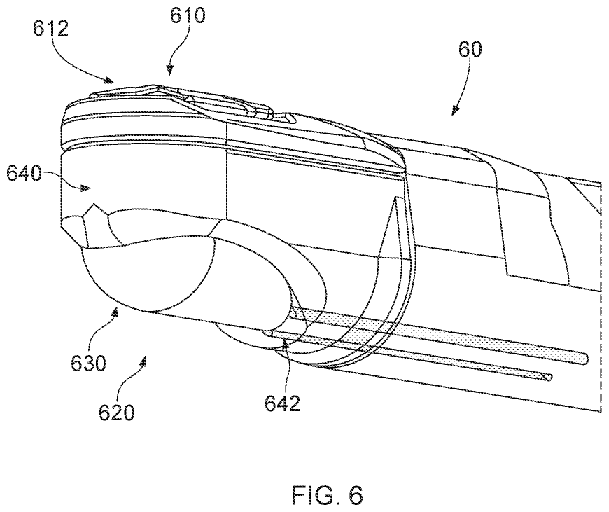

FIG. 6 shows dual-sided RF shaver combination device 60, which is much like the dual-sided RF shaver device 20 as in FIG. 2. The device has a RF side 610, comprising an active electrode area 212, and a shaver side 620 which are the same as the RF side 210 and shaver side 220 in FIG. 2. However, the arrangement in FIG. 6 differs to FIG. 2 in that the longitudinal grooves 642 are now positioned on the internal surface of the outer tubular member, stator or outer blade 640 rather than on the inner tubular member or inner blade 630, which was the case in FIG. 2.

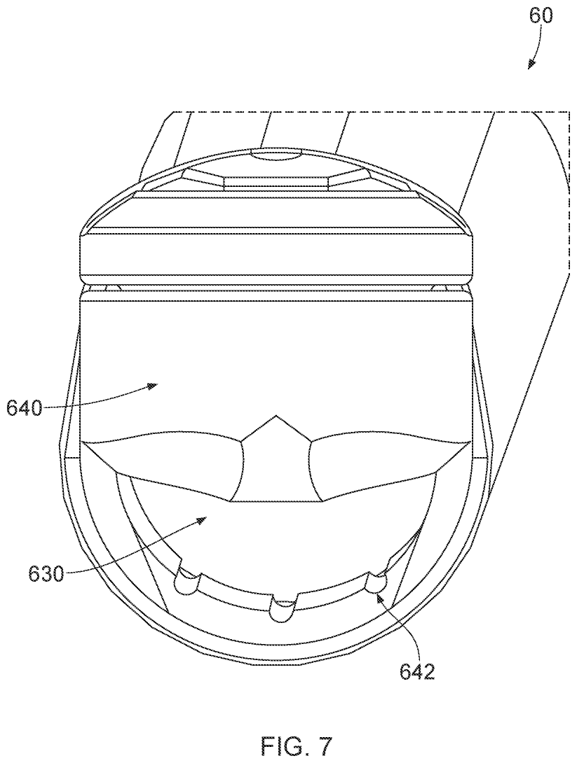

FIG. 7 shows a front perspective view of the dual-sided RF shaver combination device 60. FIG. 7 clearly shows the longitudinal grooves 642 present on the outer tubular member or stator 640. The figure shows the dual sided RF shaver combination device having three separate longitudinal grooves 642, however, it is feasible that there could be more or less longitudinal grooves 642.

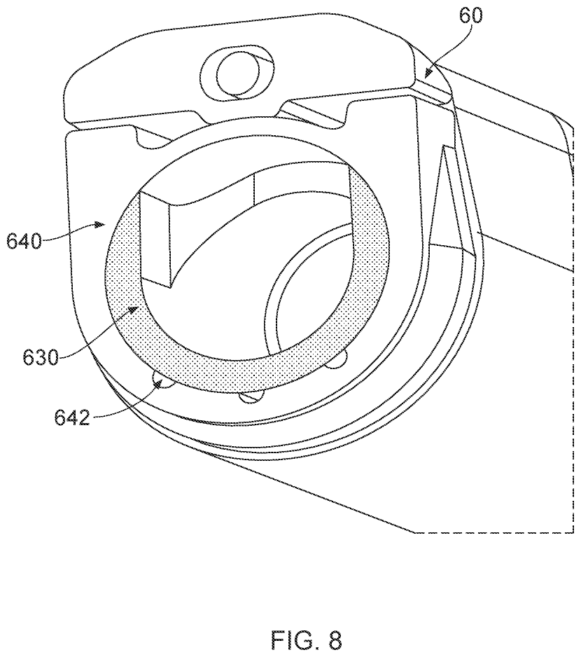

FIG. 8 shows the same dual-sided RF shaver combination device 60 as FIG. 6, but the device is shown in a partial cross-sectional arrangement to better show the longitudinal grooves 642 that are present on the outer tubular member 640. FIG. 8 shows the longitudinal grooves 642 as being dug into the inner surface of the outer tubular member or inner blade 640. Therefore, it can be seen that the longitudinal grooves 642 create passageways between the inner tubular member 630 and the outer tubular member 640, in which cool saline can pass to cool the RF suction lumen and inner tubular member 630.

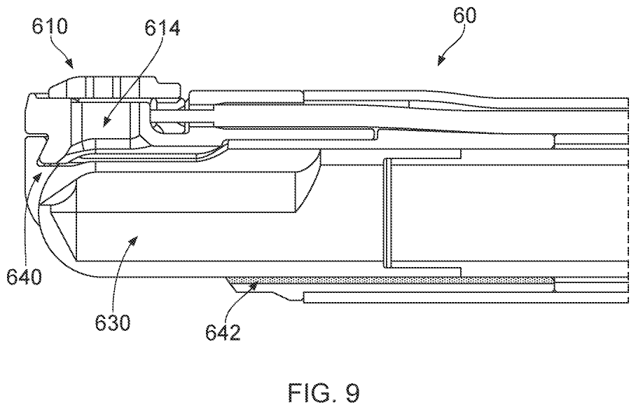

FIG. 9 shows the same dual-sided RF shaver combination device as in FIGS. 6 and 8, but the device is shown in a partial cross-sectional arrangement from the side. It can be seen that the RF side 610 comprises a RF suction pathway 614.

FIGS. 6-9 show an arrangement of the RF shaver arrangement according to a second embodiment. The key design feature of this embodiment is that the outer tubular member, stator, or outer blade 640 comprises the longitudinal grooves 642 that create a cool saline passageway in order to cool the inner tubular member 630 and RF suction lumen, that can reach high temperatures as a result of RF ablation/coagulation. In FIGS. 6-9 three longitudinal grooves 642 were depicted for example purposes, it is feasible that there could be more or less longitudinal grooves 642. An advantage of this embodiment is that the longitudinal grooves 642 are positioned on the inner surface of the stator or outer tubular member 640, as such there is no additional risk of tissue effect when shaving as this component is stationary.

In a third embodiment, the inner tubular member or inner blade and the outer tubular member or stator can both comprise longitudinal grooves as explained in the first and second embodiment. In this case, when the longitudinal grooves on the inner tubular member and the longitudinal grooves on the outer tubular member align when the inner tubular member parks in the closed position, a saline cooling passageway that is effectively twice the size of the passageways in the first and second embodiment is created. However, in another embodiment, even when the grooves on the inner and outer tubular members are not in alignment, there is still twice the cross-sectional area of suction lumen than the case when the grooves are provided on only one of the tubular members.

In a fourth embodiment, the arrangements discussed in the first, second or third embodiment can be altered to change the pathway of the longitudinal grooves. Instead of the grooves being longitudinal i.e. from distal end to the proximal end directly, the grooves could be arranged in an alternative pathway. For example, if we take the first embodiment, the longitudinal grooves 232 could be run in a spiral pathway that runs around the inner tubular member 230 from the distal end to the proximal end of the device. This would increase the potential length of the grooves even in the same sized dual-sided RF shaver combination device. This has the potential to increase the cooling capacity due to the increased length of the pathway, and therefore the increased surface area used for cooling.

The grooves discussed in the first, second, third and fourth embodiments can have a variety of cross-sectional geometries or arrangements. In FIGS. 1-9 the grooves are shown to have a semi-circular cross-sectional geometry, however, the grooves could have a square or triangle cross-sectional geometry etc. Further, there could be a plurality of small grooves or a single large/wide groove. Moreover, if there is a plurality of grooves the grooves may be spaced equidistance from one another or bunched together into smaller groups etc.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

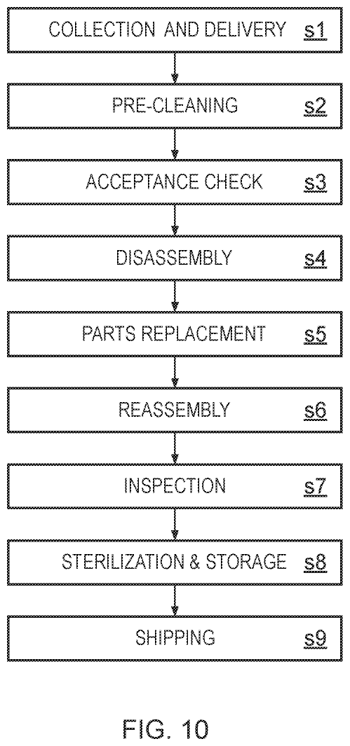

The rotary shaver arrangement described above may be disposed of after one use, or may be repeatedly used a plurality of times. In the case of a configuration that is repeatedly used a plurality of times, for example, reprocessing method shown in FIG. 10 is required. An operator collects the used rotary shaver arrangement after it has been used for treatment and transports it to a factory or the like (Step S1). Then, the operator cleans and sterilizes the collected and transported used rotary shaver arrangement (Step S2). Next, the operator performs an acceptance check of the used rotary shaver arrangement (Step S3). Subsequently, the operator disassembles the used rotary shaver arrangement (Step S4) and replaces some parts of the used rotary shaver arrangement with new parts (Step S5). After step S5, the operator assembles a new rotary shaver arrangement (Step S6). In some examples, Step S6 can include adding an identifier to indicate the device has been modified from its original condition, such as a adding a label or other marking to designate the device as reprocessed, refurbished or remanufactured. After Step S6, the operator sequentially performs an inspection (Step S7), sterilization and storage (Step S8), and shipping (Step S9) of the new rotary shaver arrangement.

Preferably, the rotary shaver arrangement described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device may also be sterilized using any other technique known in the art, including but limited to beta or gamma radiation, ethylene oxide, or steam.

Various modifications, whether by addition, substitution, or deletion will be apparent to the intended reader to provide further embodiments of the present disclosure, any and all of which are intended to be encompassed by the appended claims.

1. A rotary shaver surgical instrument, comprising:
an outer tubular member having an outer cutting window formed therein;
an inner tubular member having an inner cutting window formed therein, the inner tubular member being rotatably arranged within the outer tubular member;
at least one of the outer tubular member or the inner tubular member having one or more grooves formed in a surface thereof running from a distal end of the surgical instrument where the inner and outer cutting windows are formed to a proximal end whereby to form in use at least one suction lumen passageway between the inner tubular member and the outer tubular member.

2. A rotary shaver surgical instrument according to claim 1, wherein the one or more grooves are formed on an outer surface of the inner tubular member.

3. A rotary shaver surgical instrument according to claim 1, wherein the one or more grooves are formed on an inner surface of the outer tubular member.

4. A rotary shaver according to claim 1, wherein the one or more grooves are formed on both an outer surface of the inner tubular member, and on an inner surface of the outer tubular member.

5. A rotary shaver according to claim 4, wherein in use respective rotation positions of the inner and outer tubular members are controlled such that the one or more grooves on the outer surface of the inner tubular member align with the one or more grooves formed on the inner surface of the outer tubular member.

6. A rotary shaver according to claim 5, wherein there are a same number of grooves on both the inner and outer tubular members.

7. The rotary shaver arrangement of claim 1, wherein the grooves are semi-circular in cross-section.

8. The rotary shaver arrangement of claim 1, wherein the grooves are square edged in cross-section.

9. The rotary shaver arrangement of claim 1, wherein the grooves extend longitudinally in at least a partial straight line from the distal end of the instrument to the proximal end.

10. The rotary shaver arrangement of claim 1, wherein the grooves follow a curved path from the distal end of the instrument to the proximal end.

11. The rotary shaver arrangement of claim 10, wherein the curved path is a spiral path from the distal end of the instrument to the proximal end.

12. An end effector for an electrosurgical instrument, the end effector comprising:
a rotary shaver arrangement, comprising: an outer tubular member having an outer cutting window formed therein; an inner tubular member having an inner cutting window formed therein, the inner tubular member being rotatably arranged within the outer tubular member; at least one of the outer tubular member or the inner tubular member having one or more grooves formed in a surface thereof running from a distal end of the rotary shaver arrangement where the inner and outer cutting windows are formed to a proximal end whereby to form in use at least one suction lumen passageway between the inner tubular member and the outer tubular member; and
a radio frequency (RF) surgical instrument arrangement including an active electrode comprising a suction aperture in fluid communication with a central suction lumen.

13. The end effector of claim 12, wherein the end effector is arranged such that the RF arrangement is positioned on a first side of the end effector, and the rotary shaver arrangement is positioned such that the direction of tissue shaving of the rotary shaver arrangement is on a second side of the end effector, the second side being opposite to the first side.

14. An electrosurgical instrument comprising:
an end effector according to claim 12; and
an operative shaft having RF electrical connections operably connected to the active electrode, and drive componentry operably connected to the rotary shaver arrangement to drive the rotary shaver arrangement to operate in use.

15. An electrosurgical system, comprising:
an RF electrosurgical generator;
a suction pump; and
an electrosurgical instrument according to claim 14, the arrangement being such that in use the RF electrosurgical generator supplies an RF coagulation or ablation signal via the RF electrical connections to the active electrode and the suction pump supplies suction via the central suction lumen connecting the suction aperture located within the electrode to the suction pump.

16. A method of reprocessing a rotary shaver arrangement comprising:
an outer tubular member having an outer cutting window formed therein;
an inner tubular member having an inner cutting window formed therein, the inner tubular member being rotatably arranged within the outer tubular member;
at least one of the outer tubular member or the inner tubular member having one or more grooves formed in a surface thereof running from a distal end of the rotary shaver arrangement where the inner and outer cutting windows are formed to a proximal end whereby to form in use at least one suction lumen passageway between the inner tubular member and the outer tubular member;
the method comprising:
a) dissembling the rotary shaver arrangement;
b) cleaning the rotary shaver arrangement;
c) replacing worn parts in the rotary shaver arrangement;
d) re-assembling the rotary shaver arrangement;
e) sterilizing the re-assembled rotary shaver arrangement; and
f) packing the sterilized rotary shaver arrangement in sterile packaging.
